(19) United States
(12) Patent Application Publication
(10) Pub. No.: US 2005/0054056 A1
(43) Pub. Date: Mar. 10, 2005

(54) Variant cell surface molecule associated with cancer

(76) Inventors: Wolfgang Ebel, Philadelphia, PA (US); Luigi Grasso, Bala Cynwyd, PA (US); Nicholas C. Nicolaides, Boothwyn, PA (US); Philip M. Sass, Audubon, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/912,922

(22) Filed: Aug. 5, 2004

Related U.S. Application Data
(60) Provisional application No. 60/493,040, filed on Aug. 5, 2003.
(60) Provisional application No. 60/502,715, filed on Sep. 12, 2003.

(51) Int. Cl. G01N 33/53; C07H 21/04; C12P 21/04; C12N 5/06

(52) U.S. Cl. 435/70.21; 435/328; 530/387.3; 536/23.53

(57) ABSTRACT

The protein and nucleic acid sequences of mesovt2, specific antibodies thereto, methods for targeting and/or inhibiting the growth of cells bearing mesovt2, and methods of use of mesovt2 for diagnosing malignancy are provided. Methods of use of the mesovt2 antibodies in the treatment of certain cancers, particularly cancers that have increased cell surface expression of the mesovt2 antigen, such as pancreatic adenocarcinoma, lung carcinoma, and ovarian cancer, also are provided. The invention also relates to cells expressing the monoclonal antibodies, derivatives, and fragments.

### CROSS REFERENCE TO RELATED APPLICATIONS

This claims benefit of U.S. Provisional Application 60/493,040, filed Aug. 5, 2003, and U.S. Provisional Application 60/502,715, filed Sep. 12, 2003, each of which is incorporated herein by reference in its entirety.

### FIELD OF THE INVENTION

This invention relates to the discovery of a mesothelin variant, mesovt2, whose RNA and protein expression are associated with cancer. The invention includes uses for employing and generating the protein and nucleic acid sequences and methods for targeting and/or inhibiting the growth of cells bearing mesovt2, and methods of use for diagnosing malignancy by mesovt2. Methods of use for therapy include humoral and cellular mediated immunity by using mesovt2-specific polypeptides and derivatives thereof. Antibodies are useful in the treatment of certain cancers, particularly cancers that have increased cell surface expression of the mesovt2 antigen such as pancreatic adenocarcinoma, lung carcinoma, and ovarian cancer. The invention also relates to hybridoma cells expressing the monoclonal antibodies, antibody derivatives, such as chimeric and humanized monoclonal antibodies, antibody fragments, mammalian cells expressing the monoclonal antibodies, derivatives and fragments, and methods of detecting and treating cancer using the antibodies, derivatives and fragments specific for mesovt2.

### BACKGROUND OF THE INVENTION

The identification of cell surface antigens specific to malignant tissues offers a therapeutic approach to the treatment of cancer. While a number of cell surface antigens have been reported to be over-expressed in malignant tissues as compared to normal tissues, only a subset are specifically expressed in the malignant state (1). The identification of cell surface proteins exhibiting structural variation due to alternative splicing, aberrant post-translational modifications, or mutation add another level of specificity to target antigens associated in disease-specific tissues (1). Mesothelin is a glycosylphosphatidylinositol (GPI)-linked glycoprotein synthesized as a 69 kDa precursor and proteolytically processed into a 30 kDa NH₂-terminal secreted form and a 40 kDa membrane-bound form (2). Mesothelin has been reported to be present on normal mesothelial cells and on the surface of several tumors, including mesothelioma and ovarian cancer (2, 3, 4, 5). Mesothelin, referred to a mesothelin A herein, was identified by two groups: one group used antisera isolated from mice immunized with the ovarian cancer cell line OVCAR3 (5), and the other cloned the encoding cDNA as a megakaryocyte potentiating factor (6). Subsequently, a third group identified an alternative splice variant that results in a soluble isoform (7). Several groups have developed therapeutic approaches to use mesothelin A-specific immunotherapies for cancer; however, due to the robust expression of this antigen on normal mesothelia, severe side effects have been reported (8). Chowdhury et al. (9, 10) teaches of the use of a mesothelin A high binding affinity to single chain antibody that is conjugated to an immunotoxin derived from the Pseudomonas enterotoxin A [SS1(scFv)-PE38] which is capable of specifically killing mesothelin A-expressing cells in vitro and produces antitumor effects against ectopic s.c. cervical epidermoid carcinoma cells that have been stably transfected with the full-length mesothelin A cDNA (10).

Here we report the discovery of a common mesothelin isoform associated with cancer cell types called mesovt2. This antigen represents a distinct isoform than that reported by others (5, 6, 7) and referenced in U.S. Pat. Nos. 5,320,956; 5,525,337; 5,817,313; 6,083,502; and 6,153,430 that describe the use of antibodies and immuno-based therapies that can target the published the mesothelin A. Mesovt2 is useful for the development of specific antiserum and other immunotherapeutic strategies known to those in the art that can target malignant cell types and avoid possible toxicity by damaging normal tissues such as those of the mesothelia (8).

Administration of antibodies and immunotoxins against mesothelin A protein has been proposed as a strategy for treatment of ovarian cancer (9, 10). Due to the robust expression of this mesothelin isoform in mesothelial cells, it is likely that side effects may occur by targeting this species. The finding that mesovt2 appears to be more commonly associated in cancer cell lines and malignant tissues suggests that it may be useful for cancer-specific targeting and thereby avoid toxicity by not recognizing the mesothelin A isoform expressed in primary mesothelial cells.

A difficult problem in antibody therapy in cancer is that, often, the target of the antibody is expressed by normal tissues as well as cancerous tissues. Thus, the antibodies that are used to kill cancer cells also have a deleterious effect on normal cells. Finding unique targets or targets that are preferentially expressed in cancer tissues has proven difficult in many cancers. More effective antibody therapies for ovarian, pancreatic, lung and other mesothelin-bearing cancers that avoid the problem of reactivity with normal tissues are needed. Specific targeting of mesovt2 may avoid this problem and offer a cancer-specific therapy.

### SUMMARY OF THE INVENTION

It has been discovered that tumors that overexpress mesothelin tend to express a splice variant termed mesovt2. Without wishing to be bound by any particular theory, it is believed that the expression of mesovt2 offers a selective advantage for malignant cell types. Previously, other researchers found overexpression of mesothelin in a number of cancers; however, the isoform identified was of the mesothelin A form. Expression of mesovt2 in cancer cell lines or primary malignant cells has not been described previously. Kojima et al. (6) identified a 69 kDa protein termed megakaryocyte potentiating factor (MPF) isolated from a pancreatic cancer cell line whereby the mesovt2 variable sequences were present as part of a preprotein precursor. However, upon expression analysis of MPF, robust expression of the gene product was observed in normal lung. This finding demonstrates the utility of this invention whereby using mesovt2-specific nucleotide or protein-based probes, specific expression of the mesovt2 isoform can be determined.

The invention provides nucleotide and protein sequences and antibodies that encode for a variant mesothelin molecule called mesovt2. Mesovt2 is specifically expressed in cancer cells. Mesovt2 expression is useful for diagnosing and treating various forms of cancer. One such method for therapy involves specific antibodies wherein the antibody to mesovt2 can distinguish it from the mesothelin A isoform readily detected by mAb K1 developed by Chang et al. (3) or SS1(scFv)-PE38 developed by Chowdhury et al. (9,10) in that (a) the antibody recognizes isoforms other than that recognized by the K1 and SS1(scFv)-PE38 antibody and binds to an epitope other than the epitope recognized by mAb K1 or single chain fv SS1(scFv)-PE38; (b) the antibody can specifically recognize the mesovt2 isoform; or (c) the antibody recognizes the domains of mesovt2 which are distinct from mesothelin A.

The antibody of the invention has an affinity of at least about 1×10⁻⁷ M, 1×10⁻⁸ M, 1×10⁻⁹ M, 1×10⁻¹⁰ M, 1×10⁻¹¹ M, or 1×10⁻¹² M and recognizes an epitope on the cell surface of target cells.

The antibody of the invention may be a chimeric antibody, including, but not limited to, a human-mouse chimeric antibody. The antibody of the invention may also be a humanized antibody. The invention also provides: hybridoma cells that express the antibodies of the invention; polynucleotides that encode the antibodies of the invention; vectors comprising the polynucleotides that encode the antibodies of the invention; and expression cells comprising the vectors of the invention.

The invention also provides a method of producing an antibody that specifically binds to the mesovt2 isoform and not the mesothelin A form wherein the antibody is distinguished from mAb K1 and SS1(scFv)-PE38 in that (a) the antibody binds to an epitope other than the epitope detected by mAb K1 and SS1(scFv)-PE38 or (b) the antibody binds a specific epitope present in mesovt2. The method comprises the step of culturing the hybridoma cell that expresses an antibody of the invention or an expression cell that comprises a vector containing a polynucleotide encoding an antibody of the invention. The expression cells of the invention may be bacterial, yeast, insect, or animal cells, preferably, mammalian cells.

The invention further provides a method of inhibiting the growth of dysplastic cells associated with increased expression of mesovt2 comprising administering to a patient with such dysplastic cells a composition comprising an antibody that specifically binds to the mesovt2 isoform wherein said antibody is distinguished from mAb K1 or SS1(scFv)-PE38 in that (a) the antibody binds to an epitope other than the epitope of mAb K1 or SS1 (scFv)-PE38, (b) the antibody binds with greater affinity than mAb K1 or SS1(scFv)-PE38 to the mesovt2 isoform; or (c) the antibody out-competes mAb K1 or SS1(scFv)-PE38 for binding to said mesovt2 isoform of mesothelin. The method may be used for various dysplastic conditions, such as, but not limited to lung, ovarian, and pancreatic cancer. In preferred embodiments, the patients are human patients. In some embodiments, the antibodies are conjugated to immunotoxic agents such as, but not limited to radionuclides, toxins, and chemotherapeutic agents.

### BRIEF DESCRIPTION OF THE DRAWINGS

FIG. 1 shows the cDNA sequence of mesovt2 (SEQ ID NO:1).

FIGS. 2A and 2B show a comparison of the cDNA sequence of mesothelin A (“mesoA”) (SEQ ID NO:3), mesovt2 (SEQ ID NO:1), and the consensus sequence (SEQ ID NO:4).

FIG. 3 shows the polypeptide sequence of mesovt2 (SEQ ID NO:2).

FIG. 4 shows a comparison of the polypeptide sequences of mesothelin A (“mesoA”) (SEQ ID NO:5), mesovt2 (SEQ ID NO:2), and the consensus polypeptide sequence (SEQ ID NO:6).

FIG. 5A shows RNA expression of mesothelin in two pancreatic cancer cell lines (PAN 1 and PAN 2).

FIG. 5B shows that, despite robust expression of mesothelin at the RNA level of PAN 1 and PAN 2, K1 antibody (which is reported to recognize mesothelin) does not detect mesothelin in all cancer cells likely due to structural alterations in the mesothelin molecule.

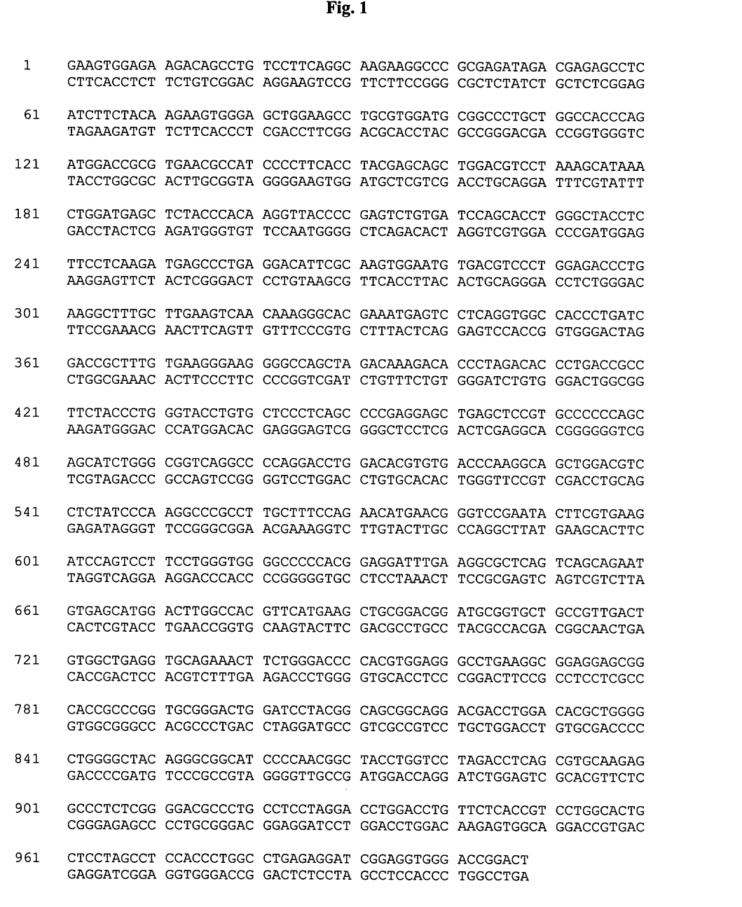

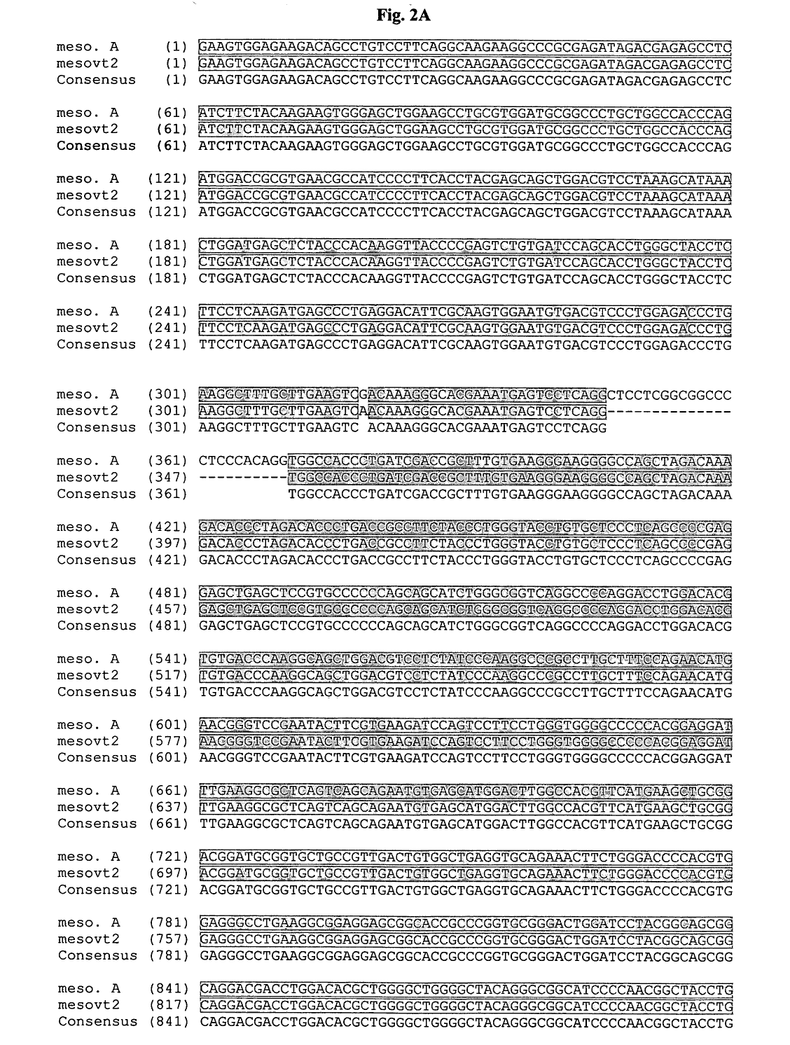

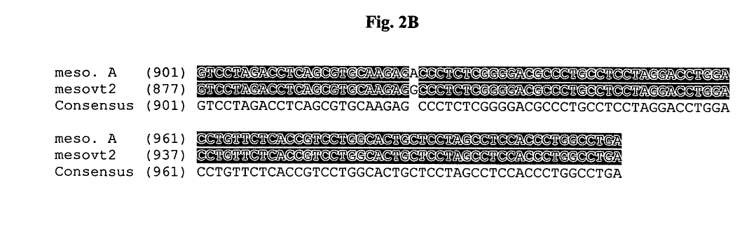

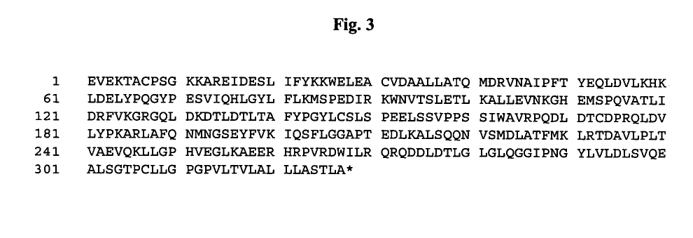

### DETAILED DESCRIPTION OF ILLUSTRATIVE EMBODIMENTS

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology known to those of skill in the art include Ausubel et al., CURRENT PROTOCOLS IN MOLECULAR BIOLOGY, John Wiley & Sons, New York (1998); Sambrook et al., MOLECULAR CLONING: A LABORATORY MANUAL, 2d ed., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991).

The invention provides a method for decreasing the growth of cancer cells and the progression of neoplastic disease using monoclonal antibodies that specifically bind to the mesovt2 isoform of mesothelin. The method of the invention may be used to modulate the growth of cancer cells and the progression of cancer in mammals, including humans. The cancer cells that may be inhibited include all cancer cells that have an increased expression of mesovt2 in relation to normal human tissues, particularly lung, ovarian, and pancreatic cancer cells.

Without wishing to be bound by any particular theory of operation, it is believed that the increased expression of mesovt2 in cancer cells results in an increased association of this isoform of mesothelin on the surface of the cells. Therefore, cancer cells have an increased expression of mesovt2 relative to normal tissues. Thus, the mesovt2 isoform of mesothelin is an ideal target for antibody or immunobased therapy in cancer.

As used herein, the term “epitope” refers to the portion of an antigen to which a monoclonal antibody specifically binds.

As used herein, the term “conformational epitope” refers to a discontinuous epitope formed by a spatial relationship between amino acids of an antigen other than an unbroken series of amino acids.

As used herein, the term “isoform” refers to a specific form of a given polypeptide.

As used herein, the term “immunobased” refers to protein-based therapies to generate immunological responses that can specifically or preferentially kill target bearing cells.

As used herein, the term “inhibition of growth of dysplastic cells in vitro” means a decrease in the number of tumor cells, in culture, by about 5%, preferably 10%, more preferably 20%, more preferably 30%, more preferably 40%, more preferably 50%, more preferably 60%, more preferably 70%, more preferably 80%, more preferably 90%, and most preferably 100%. In vitro inhibition of tumor cell growth may be measured by assays known in the art, such as the GEO cell soft agar assay.

As used herein, the term “inhibition of growth of dysplastic cells in vivo” means a decrease in the number of tumor cells, in an animal, by about 5%, preferably 10%, more preferably 20%, more preferably 30%, more preferably 40%, more preferably 50%, more preferably 60%, more preferably 70%, more preferably 80%, more preferably 90%, and most preferably 100%. In vivo modulation of tumor cell growth may be measured by assays known in the art.

As used herein, “dysplastic cells” refer to cells that exhibit abnormal growth. Dysplastic cells include, but are not limited to tumor cells, hyperplastic cells, and the like.

The term “preventing” refers to decreasing the probability that an organism contracts or develops an abnormal condition.

The term “treating” refers to having a therapeutic effect and at least partially alleviating or abrogating an abnormal condition in the organism. Treating includes maintenance of inhibited tumor growth and induction of remission.

The term “therapeutic effect” refers to the inhibition of an abnormal condition. A therapeutic effect relieves to some extent one or more of the symptoms of the abnormal condition. In reference to the treatment of abnormal conditions, a therapeutic effect can refer to one or more of the following: (a) an increase or decrease in the proliferation, growth, and/or differentiation of cells; (b) inhibition (i.e., slowing or stopping) of growth of tumor cells in vivo (c) promotion of cell death; (d) inhibition of degeneration; (e) relieving to some extent one or more of the symptoms associated with the abnormal condition; and (f) enhancing the function of a population of cells. The monoclonal antibodies and derivatives thereof described herein effectuate the therapeutic effect alone or in combination with conjugates or additional components of the compositions of the invention.

As used herein, the term “inhibits the progression of cancer” refers to an activity of a treatment that slows the modulation of neoplastic disease toward end-stage cancer in relation to the modulation toward end-stage disease of untreated cancer cells.

As used herein, the term “about” refers to an approximation of a stated value within an acceptable range. Preferably the range is +/−5% of the stated value.

As used herein, the term “neoplastic disease” refers to a condition marked by abnormal proliferation of cells of a tissue.

Antibodies

The antibodies of the invention specifically bind the mesovt2 isoform of mesothelin. In some embodiments, the antibodies bind to other forms of mesothelin. In other embodiments, the antibodies bind to an epitope other than that bound by mAb K1 or SS1(scFv)-PE38.

Preferred antibodies, and antibodies suitable for use in the method of the invention, include, for example, fully human antibodies, human antibody homologs, humanized antibody homologs, chimeric antibody homologs, Fab, Fab′, F(ab′)₂ and F(v) antibody fragments, single chain antibodies, and monomers or dimers of antibody heavy or light chains or mixtures thereof.

The antibodies of the invention may include intact immunoglobulins of any isotype including types IgA, IgG, IgE, IgD, IgM (as well as subtypes thereof). The light chains of the immunoglobulin may be kappa or lambda.

The antibodies of the invention include portions of intact antibodies that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like. Thus, antigen binding fragments, as well as full-length dimeric or trimeric polypeptides derived from the above-described antibodies, are themselves useful.

The expression cells of the invention include any insect expression cell line known, such as for example, Spodoptera frugiperda cells. The expression cell lines may also be yeast cell lines, such as, for example, Saccharomyces cerevisiae and Schizosaccharomyces pombe cells. The expression cells may also be mammalian cells such as, for example Chinese Hamster Ovary, baby hamster kidney cells, human embryonic kidney line 293, normal dog kidney cell lines, normal cat kidney cell lines, monkey kidney cells, African green monkey kidney cells, COS cells, non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TRI cells, MRC 5 cells, and FS4 cells.

A “chimeric antibody” is an antibody produced by recombinant DNA technology in which all or part of the hinge and constant regions of an immunoglobulin light chain, heavy chain, or both, have been substituted for the corresponding regions from another animal's immunoglobulin light chain or heavy chain. In this way, the antigen-binding portion of the parent monoclonal antibody is grafted onto the backbone of another species' antibody. One approach, described in EP 0239400 to Winter et al. describes the substitution one species complementarity determining regions (CDRs) for those of another species, such as substituting the CDRs from human heavy and light chain immunoglobulin variable region domains with CDRs from mouse variable region domains. These altered antibodies may subsequently be combined with human immunoglobulin constant regions to form antibodies that are human except for the substituted murine CDRs which are specific for the antigen. Methods for grafting CDR regions of antibodies may be found, for example in Riechmann et al. (1988) Nature 332:323-327 and Verhoeyen et al. (1988) Science 239:1534-1536.

Chimeric antibodies were thought to circumvent the problem of eliciting an immune response in humans than chimeric antibodies containing less murine amino acid sequences. It was found that the direct use of rodent MAbs as human therapeutic agents led to human anti-rodent antibody (“HARA”) responses which occurred in a significant number of patients treated with the rodent-derived antibody (Khazaeli, et al., (1994) Immunother. 15:42-52).

As a non-limiting example, a method of performing CDR grafting may be performed by sequencing the mouse heavy and light chains of the antibody of interest that binds to the target antigen (e.g., mesovt2) and genetically engineering the CDR DNA sequences and imposing these amino acid sequences to corresponding human V regions by site-directed mutagenesis. Human constant region gene segments of the desired isotype are added, and the “humanized” heavy and light chain genes are co-expressed in mammalian cells to produce soluble humanized antibody. A typical expression cell is a Chinese Hamster Ovary (CHO) cell. Suitable methods for creating the chimeric antibodies may be found, for example, in Jones et al. (1986) Nature 321:522-525; Riechmann (1988) Nature 332:323-327; Queen et al. (1989) Proc. Nat. Acad. Sci. USA 86:10029; and Orlandi et al. (1989) Proc. Natl. Acad. Sci. USA 86:3833.

Further refinement of antibodies to avoid the problem of HARA responses led to the development of “humanized antibodies.” Humanized antibodies are produced by recombinant DNA technology, in which at least one of the amino acids of a human immunoglobulin light or heavy chain that is not required for antigen binding has been substituted for the corresponding amino acid from a nonhuman mammalian immunoglobulin light or heavy chain. For example, if the immunoglobulin is a mouse monoclonal antibody, at least one amino acid that is not required for antigen binding is substituted using the amino acid that is present on a corresponding human antibody in that position. Without wishing to be bound by any particular theory of operation, it is believed that the “humanization” of the monoclonal antibody inhibits human immunological reactivity against the foreign immunoglobulin molecule.

Queen et al. (1989) Proc. Nat. Acad. Sci. USA 86:10029-10033 and WO 90/07861 describe the preparation of a humanized antibody. Human and mouse variable framework regions were chosen for optimal protein sequence homology. The tertiary structure of the murine variable region was computer-modeled and superimposed on the homologous human framework to show optimal interaction of amino acid residues with the mouse CDRs. This led to the development of antibodies with improved binding affinity for antigen (which is typically decreased upon making CDR-grafted chimeric antibodies). Alternative approaches to making humanized antibodies are known in the art and are described, for example, in Tempest (1991) Biotechnology 9:266-271.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The antibodies of the invention may be used alone or as immunoconjugates with a cytotoxic agent. In some embodiments, the cytotoxic agent is a radioisotope, including, but not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, and fissionable nuclides such as Boron-10 or an Actinide. In other embodiments, the cytotoxic agent is a well-known toxin and/or cytotoxic drug, including but not limited to ricin, modified Pseudomonas enterotoxin A, calicheamicin, adriamycin, 5-fluorouracil, and the like. Conjugation of antibodies and antibody fragments to such cytotoxic agents is well-known in the literature.

The antibodies of the invention include derivatives that are modified, e.g., by the covalent attachment of any type of molecule to the antibody such that covalent attachment does not prevent the antibody from binding to its epitope. Examples of suitable derivatives include, but are not limited to glycosylated antibodies and fragments, acetylated antibodies and fragments, pegylated antibodies and fragments, phosphorylated antibodies and fragments, and amidated antibodies and fragments. The antibodies and derivatives thereof of the invention may themselves by derivatized by known protecting blocking groups, proteolytic cleavage, linkage to a cellular ligand or other proteins, and the like. Further, the antibodies and derivatives thereof of the invention may contain one or more non-classical amino acids.

The invention also encompasses fully human antibodies such as those derived from peripheral blood mononuclear cells of ovarian cancer patients. Such cells may be fused with myeloma cells, for example to form hybridoma cells producing fully human antibodies against mesovt2.

Without wishing to be bound by any particular theory of operation, it is believed that the antibodies of the invention are particularly useful to bind the mesovt2 isoform of mesothelin due to a structural alteration within the protein due to the lack of 8 amino acids resulting from an alternative splicing event associated with cancer cells. This leads to a protein antigen that can be specifically recognized in cancer cells. This is an especially good feature for targeting tumors as the antibodies of the invention will bind more tightly to tumor tissue than normal tissue.

Methods of Producing Antibodies to mesovt2

Immunizing Animals

The invention also provides methods of producing monoclonal antibodies that specifically bind to the mesovt2 isoform of mesothelin. mesovt2 may be purified from cells or from recombinant systems using a variety of well-known techniques for isolating and purifying proteins. For example, but not by way of limitation, mesovt2 may be isolated based on the apparent molecular weight of the protein by running the protein on an SDS-PAGE gel and blotting the proteins onto a membrane. Thereafter, the appropriate size band corresponding to the mesovt2 of mesothelin may be cut from the membrane and used as an immunogen in animals directly, or by first extracting or eluting the protein from the membrane. As an alternative example, the protein may be isolated by size-exclusion chromatography alone or in combination with other means of isolation and purification. Other means of purification are available in such standard reference texts as Zola, MONOCLONAL ANTIBODIES: PREPARATION AND USE OF MONOCLONAL ANTIBODIES AND ENGINEERED ANTIBODY DERIVATIVES (BASICS: FROM BACKGROUND TO BENCH) Springer-Verlag Ltd., New York, 2000; BASIC METHODS IN ANTIBODY PRODUCTION AND CHARACTERIZATION, Chapter 11, “Antibody Purification Methods,” Howard and Bethell, Eds., CRC Press, 2000; ANTIBODY ENGINEERING (SPRINGER LAB MANUAL.), Kontermann and Dubel, Eds., Springer-Verlag, 2001.

One strategy for generating antibodies against mesovt2 involves immunizing animals with the recombinant form of mesovt2 or polypeptides that consist of the region specific to mesovt2. Animals so immunized will produce antibodies against the protein or polypeptide. Thus, the invention includes polyclonal sera containing antibodies that specifically bind mesovt2. Standard methods are known for creating monoclonal antibodies including, but are not limited to, the hybridoma technique (see Kohler & Milstein, (1975) Nature 256:495-497); the trioma technique; the human B-cell hybridoma technique (see Kozbor et al. (1983) Immunol. Today 4:72) and the EBV hybridoma technique to produce human monoclonal antibodies (see Cole, et al. in MONOCLONAL ANTIBODIES AND CANCER THERAPY, Alan R. Liss, Inc., 1985, pp. 77-96).

Screening for Antibody Specificity

Screening for antibodies that specifically bind to the mesovt2 isoform of mesothelin may be accomplished using an enzyme-linked immunosorbent assay (ELISA) in which microtiter plates are coated with the mesovt2 form of mesothelin. Antibodies from positively reacting clones can be further screened for reactivity in an ELISA-based assay to the mesothelin A form of mesothelin using microtiter plates coated with the mesothelin A. Clones that produce antibodies that are reactive to the mesothelin A form of mesothelin are eliminated, and clones that produce antibodies that are reactive to the mesovt2 isoform only are selected for further expansion and development.

Confirmation of reactivity of the antibodies to the mesovt2 isoform of mesothelin may be accomplished, for example, using a Western Blot assay in which protein from lung, ovarian, or pancreatic cancer cells and purified mesovt2 and mesothelin A are run on an SDS-PAGE gel under reducing and non-reducing conditions, and subsequently are blotted onto a membrane. The membrane may then be probed with the putative anti-mesovt2 antibodies. Reactivity with the mesovt2 form of mesothelin under non-reducing conditions and not the mesothelin A form of mesothelin (under reducing or non-reducing conditions) confirms specificity of reactivity for the mesovt2 isoform.

The antibodies and derivatives thereof of the invention have binding affinities that include a dissociation constant (K_{d}) of less than 1×10⁻². In some embodiments, the K_{d} is less than 1×10⁻³. In other embodiments, the K_{d} is less than 1×10⁻⁴. In some embodiments, the K_{d} is less than 1×10⁻⁵. In still other embodiments, the K_{d} is less than 1×10⁻⁶. In other embodiments, the K_{d} is less than 1×10⁻⁷. In other embodiments, the K_{d} is less than 1×10⁻⁸. In other embodiments, the K_{d} is less than 1×10⁻⁹. In other embodiments, the K_{d} is less than 1×10⁻¹⁰. In still other embodiments, the K_{d} is less than 1×10⁻¹¹. In some embodiments, the K_{d} is less than 1×10⁻². In other embodiments, the K_{d} is less than 1×10⁻¹³. In other embodiments, the K_{d} is less than 1×10⁻¹⁴. In still other embodiments, the K_{d} is less than 1×10⁻¹⁵.

Production of Antibodies

Antibodies of the invention may be produced in vivo or in vitro. For in vivo antibody production, animals are generally immunized with an immunogenic portion of mesovt2 (preferably the region specific for mesovt2).

In one embodiment, the immunogen used for immunizing animals or immunizing cells in vitro is a polypeptide comprising the amino acid sequence of VNKGHEMSPQVATLIDRFVKGRGQLDK (SEQ ID NO:7) or immunogenic portions thereof. The immunogenic portion comprises at least 10-27 contiguous amino acids of SEQ ID NO:7. In some embodiments, the immunogenic portion comprises at least 10 contiguous amino acids of SEQ ID NO:7, in other embodiments, the immunogenic portion comprises at least 15 contiguous amino acids of SEQ ID NO:7, in other embodiments, the immunogenic portion comprises at least 17 contiguous amino acids of SEQ ID NO:7, in other embodiments, the immunogenic portion comprises at least 20 contiguous amino acids of SEQ ID NO:7, in other embodiments, the immunogenic portion comprises at least 24 contiguous amino acids of SEQ ID NO:7, in other embodiments, the immunogenic portion consists of the entire 27 contiguous amino acids of SEQ ID NO:7.

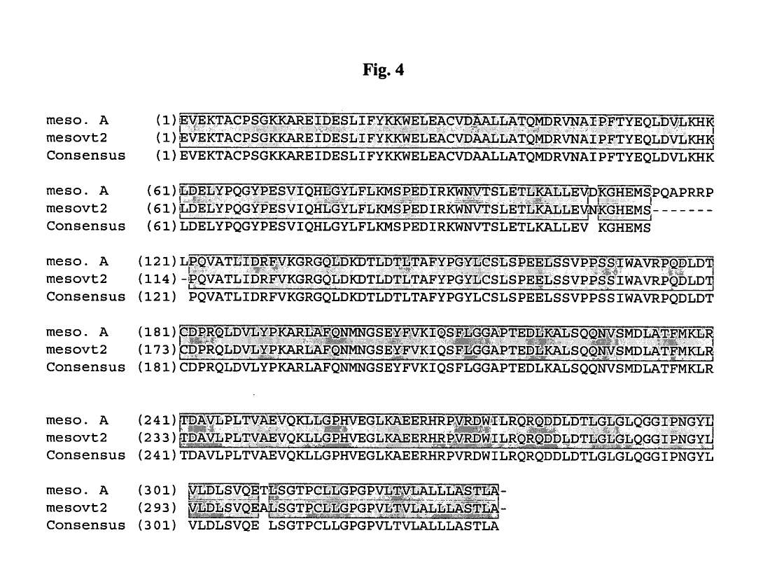

In a specific embodiment, a peptide, designated pMESO2, having the amino acid sequence of NKGHEMSPQVATLID (SEQ ID NO:12) is synthesized. This peptide spans the junction in which the deletion of amino acids occurs when comparing mesothelin A to mesovt2 (see FIG. 4). The junction occurs between the serine and proline in SEQ ID NO:12; that is NKGHEMS//PQVATLID. In addition, other peptides may be synthesized for other portions of the mesovt2 molecule such as pMESO1: EVEKTACPSGKKARE (SEQ ID NO:13); pMESO 3: RFVKGRGQLDKDTLD (SEQ ID NO:14); and pMESO4: HVEGLKAEERHRPVR (SEQ ID NO:15). For experiments in which tetanus toxoid (TT) is fused to the mesovt2 peptides or protein, one may also develop antibodies to TT by immunizing animals with a peptide derived from the TT amino acid sequence, such as pTT: QYIKANSKFIGITEL (SEQ ID NO:16). The peptides pMESO 1-4 and pTT have the characteristics shown in Table 1:

TABLE 1

| | Amino acid | Molecular | |
|---|---|---|---|
| Peptide | location in protein | weight (kD) | pI |
| pMESO1 | 20-34 | 1.634 | 8 |
| pMESO2 | 126-140 | 1.640 | 5.2 |
| pMESO3 | 141-155 | 1.749 | 9.89 |
| pMESO4 | 270-284 | 1.658 | 10 |
| pTT | 830-844 | 1.725 | 8.50 |

In some embodiments, the animals or in vitro system cells are immunized with an immunogenic portion of SEQ ID NO:12, SEQ ID NO:13, SEQ ID NO:14, SEQ ID NO:15 or SEQ ID NO:16. The immunogenic portion comprises at least 10 contiguous amino acids of SEQ ID NO:12, SEQ ID NO:13, SEQ ID NO:14, SEQ ID NO:15 or SEQ ID NO:16. In some embodiments, the immunogenic portion comprises at least 11 contiguous amino acids of SEQ ID NO:12, SEQ ID NO:13, SEQ ID NO:14, SEQ ID NO:15 or SEQ ID NO:16; in other embodiments, the immunogenic portion comprises at least 12 contiguous amino acids of SEQ ID NO:12, SEQ ID. NO:13, SEQ ID NO:14, SEQ ID NO:15 or SEQ ID NO:16; in other embodiments, the immunogenic portion comprises at least 13 contiguous amino acids of SEQ ID NO:12, SEQ ID NO:13, SEQ ID NO:14, SEQ ID NO:15 or SEQ ID NO:16; in other embodiments, the immunogenic portion comprises at least 14 contiguous amino acids of SEQ ID NO:12, SEQ ID NO:13, SEQ ID NO:14, SEQ ID NO:15 or SEQ ID NO:16.

Antibodies raised against pMESO 2 (SEQ ID NO:12) are expected to recognize and specifically bind to mesovt2 and not mesothelin A. Without wishing to be bound by any particular theory of operation, it is believed that such antibodies are specific for mesovt2 and will specifically bind to tumors such as pancreatic adenocarcinoma, lung carcinoma, and ovarian cancers. As such, these antibodies are particularly useful in the treatment of tumors. The antibodies may be fully human antibodies, chimerized antibodies, humanized antibodies, single chain antibodies, Fab, Fab′, F(ab′)₂, F(v) antibody fragments or polyclonal antibodies.

The antigen is generally combined with an adjuvant to promote immunogenicity. Adjuvants vary according to the species used for immunization. Examples of adjuvants include, but are not limited to: Freund's complete adjuvant (“FCA”), Freund's incomplete adjuvant (“FIA”), mineral gels (e.g., aluminum hydroxide), surface active substances (e.g., lysolecithin, pluronic polyols, polyanions), peptides, oil emulsions, keyhole limpet hemocyanin (“KLH”), dinitrophenol (“DNP”), and potentially useful human adjuvants such as Bacille Calmette-Guerin (“BCG”) and Corynebacterium parvum. Such adjuvants are also well known in the art.

Immunization may be accomplished using well-known procedures. The dose and immunization regimen will depend on the species of mammal immunized, its immune status, body weight, and/or calculated surface area, etc. Typically, blood serum is sampled from the immunized mammals and assayed for anti-mesovt2 antibodies using appropriate screening assays as described below, for example.

Splenocytes from immunized animals may be immortalized by fusing the splenocytes (containing the antibody-producing B cells) with an immortal cell line such as a myeloma line. Typically, the myeloma cell line is from the same species as the splenocyte donor. In one embodiment, the immortal cell line is sensitive to culture medium containing hypoxanthine, aminopterin, and thymidine (“HAT medium”). In some embodiments, the myeloma cells are negative for Epstein-Barr virus (EBV) infection. In preferred embodiments, the myeloma cells are HAT-sensitive, EBV negative, and Ig expression negative. Any suitable myeloma may be used. Murine hybridomas may be generated using mouse myeloma cell lines (e.g., the P3-NS1/1-Ag4-1, P3-x63-Ag8.653, or Sp2/O-Ag14 myeloma lines). These murine myeloma lines are available from the ATCC. These myeloma cells are fused to the donor splenocytes in polyethylene glycol (“PEG”), preferably 1500 molecular weight polyethylene glycol (“PEG 1500”). Hybridoma cells resulting from the fusion are selected in HAT medium which kills unfused and unproductively fused myeloma cells. Unfused splenocytes die over a short period of time in culture. In some embodiments, the myeloma cells do not express immunoglobulin genes.

Hybridomas producing a desired antibody which are detected by screening assays such as those described below, may be used to produce antibodies in culture or in animals. For example, the hybridoma cells may be cultured in a nutrient medium under conditions and for a time sufficient to allow the hybridoma cells to secrete the monoclonal antibodies into the culture medium. These techniques and culture media are well known by those skilled in the art. Alternatively, the hybridoma cells may be injected into the peritoneum of an unimmunized animal. The cells proliferate in the peritoneal cavity and secrete the antibody, which accumulates as ascites fluid. The ascites fluid may be withdrawn from the peritoneal cavity with a syringe as a rich source of the monoclonal antibody.

Another non-limiting method for producing human antibodies is described in U.S. Pat. No. 5,789,650 which describes transgenic mammals that produce antibodies of another species (e.g., humans) with their own endogenous immunoglobulin genes being inactivated. The genes for the heterologous antibodies are encoded by human immunoglobulin genes. The transgenes containing the unrearranged immunoglobulin-encoding regions are introduced into a non-human animal. The resulting transgenic animals are capable of functionally rearranging the transgenic immunoglobulin sequences and producing a repertoire of antibodies of various isotypes encoded by human immunoglobulin genes. The B-cells from the transgenic animals are subsequently immortalized by any of a variety of methods, including fusion with an immortalizing cell line (e.g., a myeloma cell).

Antibodies against mesovt2 may also be prepared in vitro using a variety of techniques known in the art. For example, but not by way of limitation, fully human monoclonal antibodies against mesovt2 may be prepared by using in vitro-primed human splenocytes (Boerner et al. (1991) J. Immunol. 147:86-95).

Alternatively, for example, the antibodies of the invention may be prepared by “repertoire cloning” (Persson et al. (1991) Proc. Nat. Acad. Sci. USA 88:2432-2436; and Huang and Stollar (1991) J. Immunol. Methods 141:227-236). Further, U.S. Pat. No. 5,798,230 describes preparation of human monoclonal antibodies from human B antibody-producing B cells that are immortalized by infection with an Epstein-Barr virus that expresses Epstein-Barr virus nuclear antigen 2 (EBNA2). EBNA2, required for immortalization, is then inactivated resulting in increased antibody titers.

In another embodiment, antibodies against mesovt2 are formed by in vitro immunization of peripheral blood mononuclear cells (“PMBCs”). This may be accomplished by any means known in the art, such as, for example, using methods described in the literature (Zafiropoulos et al. (1997) J. Immunological Methods 200:181-190).

In one embodiment of the invention, the procedure for in vitro immunization is supplemented with directed evolution of the hybridoma cells in which a dominant negative allele of a mismatch repair gene such as PMS1, PMS2, PMS2-134, PMSR2, PMSR3, MLH1, MLH2, MLH3, MLH4, MLH5, MLH6, PMSL9, MSH1, and MSH2 is introduced into the hybridoma cells after fusion of the splenocytes, or to the myeloma cells before fusion. Cells containing the dominant negative mutant will become hypermutable and accumulate mutations at a higher rate than untransfected control cells. A pool of the mutating cells may be screened for clones that produce higher affinity antibodies, or that produce higher titers of antibodies, or that simply grow faster or better under certain conditions. The technique for generating hypermutable cells using dominant negative alleles of mismatch repair genes is described in U.S. Pat. No. 6,146,894, issued Nov. 14, 2000. Alternatively, mismatch repair may be inhibited using the chemical inhibitors of mismatch repair described by Nicolaides et al. in WO 02/054856 “Chemical Inhibitors of Mismatch Repair” published Jul. 18, 2002. The technique for enhancing antibodies using the dominant negative alleles of mismatch repair genes or chemical inhibitors of mismatch repair may be applied to mammalian expression cells expressing cloned immunoglobulin genes as well. Cells expressing the dominant negative alleles can be “cured” in that the dominant negative allele can be turned off if inducible, eliminated from the cell, and the like, such that the cells become genetically stable once more and no longer accumulate mutations at the abnormally high rate.

Methods of Reducing the Growth of Tumor Cells

The methods of the invention are suitable for use in humans and non-human animals identified as having a neoplastic condition associated with an increased expression of mesovt2. Non-human animals which benefit from the invention include pets, exotic (e.g., zoo animals) and domestic livestock. Preferably the non-human animals are mammals.

The invention is suitable for use in a human or animal patient that is identified as having a dysplastic disorder that is marked by increased expression of mesovt2 in the neoplasm in relation to normal tissues. Once such a patient is identified as in need of treatment for such a condition, the method of the invention may be applied to effect treatment of the condition. Tumors that may be treated include, but are not limited to ovarian tumors, pancreatic tumors, lung tumors, fallopian tube tumors, uterine tumors, and certain leukemia cells.

The antibodies and derivatives thereof for use in the invention may be administered orally in any acceptable dosage form such as capsules, tablets, aqueous suspensions, solutions or the like. The antibodies and derivatives thereof may also be administered parenterally. That is via the following routes of administration: subcutaneous, intravenous, intramuscular, intra-articular, intra-synovial, intrasternal, intranasal, topically, intrathecal, intrahepatic, intralesional, and intracranial injection or infusion techniques. Generally, the antibodies and derivatives will be provided as an intramuscular or intravenous injection.

The antibodies and derivatives of the invention may be administered alone of with a pharmaceutically acceptable carrier, including acceptable adjuvants, vehicles and excipients.

The effective dosage will depend on a variety of factors and it is well within the purview of a skilled physician to adjust the dosage for a given patient according to various parameters such as body weight, the goal of treatment, the highest tolerated dose, the specific formulation used, the route of administration and the like. Generally, dosage levels of between about 0.001 and about 100 mg/kg body weight per day of the antibody or derivative thereof are suitable. In some embodiments, the dose will be about 0.1 to about 50 mg/kg body weight per day of the antibody or derivative thereof. In other embodiments, the dose will be about 0.1 mg/kg body weight/day to about 20 mg/kg body weight/day. In still other embodiments, the dose will be about 0.1 mg/kg body weight/day to about 10 mg/kg body weight/day. Dosing may be as a bolus or an infusion. Dosages may be given once a day or multiple times in a day. Further, dosages may be given multiple times of a period of time. In some embodiments, the doses are given every 1-14 days. In some embodiments, the antibodies or derivatives thereof are given as a dose of about 3 to 1 mg/kg i.p. In other embodiments, the antibodies of derivatives thereof are provided at about 5 to 12.5 mg/kg i.v. In still other embodiments, the antibodies or derivatives thereof are provided such that a plasma level of at least about 1 ug/ml is maintained.

Effective treatment may be assessed in a variety of ways. In one embodiment, effective treatment is determined by a slowed progression of tumor growth. In other embodiments, effective treatment is marked by shrinkage of the tumor (i.e., decrease in the size of the tumor). In other embodiments, effective treatment is marked by inhibition of metastasis of the tumor. In still other embodiments, effective therapy is measured by increased well-being of the patient including such signs as weight gain, regained strength, decreased pain, thriving, and subjective indications from the patient of better health.

The following Examples are provided to illustrate the present invention, and should not be construed as limiting thereof.

### EXAMPLES

### Example 1

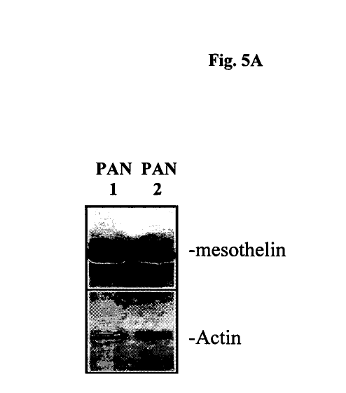

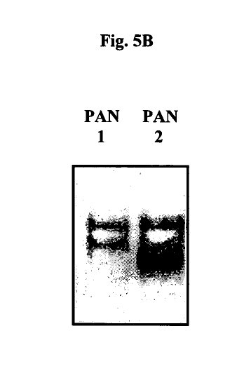

Lack of binding of the monoclonal antibody K1 in mesothelin expressing pancreatic cancer cell lines expressing mesovt2 was shown by Western blot (FIG. 5B). Briefly, pancreatic cancer cells are isolated and lysed for RNA and protein extraction. cDNA from OVCAR-3 and KB cells was obtained with the Pierce DIRECTEXPRESS RT-PCR kit

The cDNA encoding the 40 kDa mesothelin portion was amplified using specific primers (numbering is based on GenBank accession # U40434):

(SEQ ID NO:8)

| | |
|---|---|
| Mesothelin-2079-R | 5′ AGTTCTCTTGGGGTGGAACGGGGAT 3′ |

(SEQ ID NO:9)

| | |
|---|---|
| Mesothelin-975-F | 5′ GCGGGAAGTGGAGAAGACAGCCTGT 3′ |

For subcloning, primers were engineered to incorporate a BsrGI sequence at the 5′ end and a 6 histidine residue coding sequence and XbaI sequence at the 3′ end of the amplicon:

| | |
|---|---|
| Mesothelin-40 kDa-fusion-BsrGI-F | |
| 5′-GATCTGTACACAGCGAAGTGGAGAAGACAGCCTGT-3′ | (SEQ ID NO:10) |
| Mesothelin-40 kDa-6His-XbaI-R | |
| 5′-GATCTCTAGATATCAATGGTGATGGTGATGATGCA | (SEQ ID NO:11) |
| TGCCCTGTAGCCCCAGCCCCAGCGT-3′ | |

Amplicons were captured by cloning into pGEM-T-Easy and sequenced with M13F and M13R primers.

Using the Pierce DIRECTEXPRESS kit, 67 ul of a dH2O/TAQ mixture was aliquoted into each sample along with appropriate gene-specific primers that target sequences contained within the mesothelin cDNA or β-actin. Amplifications were carried out using the following amplification conditions: 94° C. for 30 sec, 55° C. for 30 sec and 72° C. for 1.5 min. PCR products were analyzed on 2% agarose gels and visualized by ethidium bromide staining and uv evaluation. The results are shown in FIG. 5A.

Western blots weare performed to determine ability of antibodies to recognize multiple isoforms of mesothelin. Briefly, cells were harvested and lysed in RIPA buffer as described by the manufacturer (Novex). Insoluble material was removed by centrifugation and the total protein of the supernate was determined using a BioRad protein Assay. In different experiments, either 5 ug or 20 ug of protein was run on a 4-12% Bis-Tris gel (MES) under non-reducing conditions. The electrophoresed protein was transferred to a PVDF membrane. The membrane was blocked in Blotto (5% milk, 0.05% TBS-T). A 1:1000 dilution of K1 Mab (Novus) was added directly to the Blotto blocking solution as the primary antibody, and the membrane was incubated overnight. The membrane was washed in three times (5 min. each) with 0.05% TBS-T and the secondary antibody (horseradish peroxidase labeled goat α-mouse IgG (heavy and light chains)) in Blotto blocking solution was added. The membrane was washed three times (20 min., 15 min., 15 min.) with 0.05% TBS-T. The membrane was developed using Super Signal West Pico ECL reagent. The results are shown in FIG. 5B. The results indicate that certain tumors that overexpress mesothelin favor the production of mesovt2 over the mesothelin A. This finding can be exploited by monoclonal antibodies that specifically recognize the mesovt2 isoform of mesothelin for the destruction of tumor tissue, while leaving normal tissue (which generally expresses the mesothelin A form of mesothelin) unscathed.

Each of the following references is hereby incorporated by reference in its entirety:
1. Iacobuzio-Donahue et al. (2003) “Exploration of global gene expression patterns in pancreatic adenocarcinoma using cDNA microarrays” Am. J. Pathol. 162:1151-1162.
2. Yamaguchi, et al. (1994) “A novel cytokine exhibiting megakaryocyte potentiating activity from a human pancreatic tumor cell line HPC-Y5” J. Biol. Chem. 269:805-808.
3. Chang, et al. (1992) “Monoclonal antibody K1 reacts with epithelial mesothelioma but not lung adenocarcinoma” Am. J. Surg. Pathol. 16:259-268.
4. Chang and Pastan (1996) “Molecular cloning of the mesothelin, a differentiation antigen present on mesothelium, mesotheliomas and ovarian cancers” Proc. Natl. Acad. Sci. USA 93:136-140.
5. Frierson, et al. (2003) “Large-scale molecular and tissue microarray analysis of mesothelin expression in common human carcinomas” Hum. Pathol. 34:605-609.
6. Kojima, et al. (1995) “Molecular cloning and expression of megakaryocyte potentiating factor cDNA” i J. Biol. Chem. 270:21984-21990.
7. Scholler, et al. (1999) “Soluble member(s) of the mesothelin/megakaryocyte potentiating factor family are detectable in sera from patients with ovarian carcinoma” Proc. Natl. Acad. Sci. USA 96:11531-11536.
8. Ordonez (2003) “Value of mesothelin immunostaining in the diagnosis of mesothelioma” Mod. Pathol. 16:192-197.
9. Chowdhury, et al. (1997) “Isolation of anti-mesothelin antibodies from a phage display library” Mol. Immunol. 34:9-20.
10. Chowdhury, et al. (1998) “Isolation of a high-affinity stable single-chain Fv specific for mesothelin from DNA-immunized mice by phage display and construction of a recombinant immunotoxin with anti-tumor activity” Proc. Natl. Acad. Sci. USA 95:669-674.

1. An antibody that specifically binds to the mesovt2 isoform (SEQ ID NO:2) of mesothelin wherein said antibody is distinguished from mAb K1 and SS1(scFv)-PE38 in that (a) said antibody binds to an epitope other than the epitope of mAb K1 and SS1(scFv)-PE38; (b) said antibody binds with greater affinity than mAb mAb K1 and SS1(scFv)-PE38; or (c) said antibody out-competes mAb K1 and SS1(scFv)-PE38 for binding to said mesovt2 form of mesothelin.

2. The antibody of claim 1 wherein the affinity of said antibody is at least about 1×10⁻⁷ M.

3. The antibody of claim 1 wherein the affinity of said antibody is at least about 1×10⁻⁸ M.

4. The antibody of claim 1 wherein the affinity of said antibody is at least about 1×10⁻⁹ M.

5. The antibody of claim 1 wherein the affinity of said antibody is at least about 1×10⁻¹⁰ M.

6. The antibody of claim 1 wherein said epitope is a disulfide-dependent epitope.

7. The antibody of claim 1 wherein said antibody is a chimeric antibody.

8. The antibody of claim 7 wherein said chimeric antibody is a human-mouse chimeric antibody.

9. The antibody of claim 1 wherein said antibody is a humanized antibody.

10. The antibody of claim 1 wherein said antibody is a fully human antibody.

11. A hybridoma cell that expresses the antibody of claim 1.

12. A polynucleotide encoding the antibody of claim 1.

13. A vector comprising the polynucleotide of claim 12.

14. An expression cell comprising the vector of claim 13.

15. A method of producing an antibody that specifically binds to the mesovt2 isoform of mesothelin wherein said antibody is distinguished from mAb K1 and SS1(scFv)-PE38 in that (a) said antibody binds to an epitope other than the epitope of mAb K1 and SS1(scFv)-PE38; (b) said antibody binds with greater affinity than mAb K1 and SS1(scFv)-PE38; or (c) said antibody out-competes mAb K1 and SS1(scFv)-PE38 for binding to said mesovt2 isoform of mesothelin, said method comprising the step of culturing the hybridoma of claim 11.

16. A method of producing an antibody that specifically binds to the mesovt2 isoform of mesothelin wherein said antibody is distinguished from mAb K1 and SS1(scFv)-PE38 in that (a) said antibody binds to an epitope other than the epitope of mAb K1 and SS1(scFv)-PE38; (b) said antibody binds with greater affinity than mAb K1 and SS1(scFv)-PE38; or (c) said antibody out-competes mAb K1 and SS1(scFv)-PE38 for binding to said mesovt2 form of mesothelin, said method comprising the step of culturing the expression cell of claim 14.

17. The expression cell of claim 14 wherein said cell is a mammalian cell.

18. The method of claim 16 wherein said expression cell is a mammalian cell.

19. A method of producing an antibody that specifically binds to the mesovt2 isoform of mesothelin wherein said antibody is distinguished from mAb K1 and SS1(scFv)-PE38 in that (a) said antibody is generated using a polypeptide comprising the amino acid sequence of SEQ ID NO:7, or antigenic fragments thereof, as antigen.

20. The method of claim 19 wherein said antibody is generated using a polypeptide consisting of the amino acid sequence of SEQ ID NO:7.

21. A method of inhibiting the growth of dysplastic cells associated with increased expression of mesovt2 comprising administering to a patient with such dysplastic cells a composition comprising an antibody that specifically binds to the mesovt2 isoform of mesothelin wherein said antibody is distinguished from mAb K1 and SS1(scFv)-PE38 in that (a) said antibody binds to an epitope other than the epitope of mAb K1 and SS1(scFv)-PE38, (b) said antibody binds with greater affinity than mAb K1 and SS1(scFv)-PE38; or (c) said antibody out-competes mAb K1 and SS1(scFv)-PE38 for binding to said mesovt2 isoform of mesothelin.

22. The method of claim 21 wherein said antibody is a monoclonal antibody.

23. The method of claim 21 wherein said dysplastic cells are lung, ovarian or pancreatic cancer cells.

24. The method of claim 21 wherein said patient is a human patient.

25. The method of claim 21 wherein said antibody is conjugated to a chemotherapeutic agent.

26. The method of claim 25 wherein said chemotherapeutic agent is a radionuclide.

27. The method of claim 26 wherein said radionuclide is selected from the group consisting of Lead-212, Bismuth-212, Astatine-211, Iodine-131, Scandium-47, Rhenium-186, Rhenium-188, Yttrium-90, Iodine-123, Iodine-125, Bromine-77, Indium-111, Boron-10 and Actinide.

28. The method of claim 25 wherein said chemotherapeutic agent is selected from the group consisting of ricin, modified Pseudomonas enterotoxin A, calicheamicin, adriamycin, and 5-fluorouracil.

29. The antibody of claim 28 wherein antibody is conjugated to a chemotherapeutic agent.

30. The antibody of claim 29 wherein said chemotherapeutic agent is a radionuclide.

31. The antibody of claim 30 wherein said radionuclide is selected from the group consisting of Lead-212, Bismuth-212, Astatine-211, Iodine-131, Scandium-47, Rhenium-186, Rhenium-188, Yttrium-90, Iodine-123, Iodine-125, Bromine-77, Indium-111, Boron-10 and Actinide.

32. The antibody of claim 25 wherein said chemotherapeutic agent is selected from the group consisting of ricin, modified Pseudomonas enterotoxin A, calicheamicin, adriamycin, and 5-fluorouracil.

33. A method of producing a vaccine antigen that specifically binds to the mesovt2 isoform of mesothelin wherein said antigen is distinguished from mesothelin A in that (a) said T-cells binds to an epitope specific to mesovt2.

34. The antibody of claim 1 wherein said antibody specifically binds to an epitope on mesovt2 comprising an amino acid sequence comprising at least 10 consecutive amino acids of SEQ ID NO:12.

35. The antibody of claim 34 wherein said epitope comprises at least 11 consecutive amino acids of SEQ ID NO:12.

36. The antibody of claim 34 wherein said epitope comprises at least 12 consecutive amino acids of SEQ ID NO:12.

37. The antibody of claim 34 wherein said epitope comprises at least 13 consecutive amino acids of SEQ ID NO:12.

38. The antibody of claim 34 wherein said epitope comprises at least 14 consecutive amino acids of SEQ ID NO:12.

39. The antibody of claim 34 wherein said epitope comprises the amino acid sequence of SEQ ID NO:12.

40. A hybridoma cell that produces an antibody of claim 34.

41. A polyclonal antibody preparation comprising antibodies that specifically bind to an epitope of mesovt2 comprising the amino acid sequence of SEQ ID NO:12.

42. A method of producing an antibody that specifically binds to the mesovt2 isoform of mesothelin wherein said antibody is distinguished from mAb K1 and SS1(scFv)-PE38 in that (a) said antibody is generated using a polypeptide comprising the amino acid sequence of SEQ ID NO:12, or antigenic fragments thereof, as antigen.

43. The method of claim 19 wherein said antibody is generated using a polypeptide consisting of the amino acid sequence of SEQ ID NO:12.

44. A method of inhibiting the growth of dysplastic cells associated with increased expression of mesovt2 comprising administering to a patient with such dysplastic cells a composition comprising an antibody that specifically binds to the mesovt2 isoform of mesothelin wherein said antibody is distinguished from mAb K1 and SS1(scFv)-PE38 in that said antibody binds to an epitope of mesovt2 comprising an amino acid sequence comprising at least 10 consecutive amino acids of SEQ ID NO:12.

45. The method of claim 44 wherein said antibody binds to an epitope of mesovt2 comprising the amino acid sequence of SEQ ID NO:12.


SEQUENCE LISTING (16 sequences)

SEQ ID NO: 1 - DNA, 1008 bp, Homo sapiens
gaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagaggccctctcggggacgccctgcctcctaggacctggacctgttctcaccgtcctggcactgctcctagcctccaccctggcctgagaggatcggaggtgggaccggact

SEQ ID NO: 2 - protein, 327 aa, Homo sapiens
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSVQEALSGTPCLLGPGPVLTVLALLLASTLA

SEQ ID NO: 3 - DNA, 1008 bp, Homo sapiens
gaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcgacaaagggcacgaaatgagtcctcaggctcctcggcggcccctcccacaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagagaccctctcggggacgccctgcctcctaggacctggacctgttctcaccgtcctggcactgctcctagcctccaccctggcctga

SEQ ID NO: 4 - DNA, 984 bp, Artificial Sequence
  Consensus Sequence
gaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcracaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagagrccctctcggggacgccctgcctcctaggacctggacctgttctcaccgtcctggcactgctcctagcctccaccctggcctga

SEQ ID NO: 5 - protein, 335 aa, Homo sapiens
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVDKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSVQETLSGTPCLLGPGPVLTVLALLLASTLA

SEQ ID NO: 6 - protein, 327 aa, Artificial Sequence
  Consensus Sequence
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVBKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSVQEXLSGTPCLLGPGPVLTVLALLLASTLA

SEQ ID NO: 7 - protein, 27 aa, Artificial Sequence
  Synthetic Peptide
VNKGHEMSPQVATLIDRFVKGRGQLDK

SEQ ID NO: 8 - DNA, 25 bp, Artificial Sequence
  Oligonucleotide primer
agttctcttggggtggaacggggat

SEQ ID NO: 9 - DNA, 25 bp, Artificial Sequence
  Oligonucleotide primer
gcgggaagtggagaagacagcctgt

SEQ ID NO: 10 - DNA, 35 bp, Artificial Sequence
  Oligonucleotide primer
gatctgtacacagcgaagtggagaagacagcctgt

SEQ ID NO: 11 - DNA, 60 bp, Artificial Sequence
  Oligonucleotide primer
gatctctagatatcaatggtgatggtgatgatgcatgccctgtagccccagccccagcgt

SEQ ID NO: 12 - protein, 15 aa, Artificial Sequence
  Synthetic Construct - pMESO2
NKGHEMSPQVATLID

SEQ ID NO: 13 - protein, 15 aa, Artificial Sequence
  Synthetic Construct - pMESO1
EVEKTACPSGKKARE

SEQ ID NO: 14 - protein, 15 aa, Artificial Sequence
  Synthetic Construct - pMESO3
RFVKGRGQLDKDTLD

SEQ ID NO: 15 - protein, 15 aa, Artificial Sequence
  Synthetic Construct - pMESO4
HVEGLKAEERHRPVR

SEQ ID NO: 16 - protein, 15 aa, Artificial Sequence
  Synthetic Construct - pTT
QYIKANSKFIGITEL